(19) United States
(12) Patent Application Publication
(10) Pub. No.: US 2003/0014107 A1
(43) Pub. Date: Jan. 16, 2003

(54) Multifocal phakic intraocular lens

(76) Inventor: Michael Reynard, Santa Monica, CA (US)

Correspondence Address: ALBERT O COTA, 5460 WHITE OAK AVE, SUITE A-331, ENCINO, CA, 91316, US

(21) Appl. No.: 09/892,607

(22) Filed: Jun. 28, 2001

(51) Int. Cl. A61F 2/16

(52) U.S. Cl. 623/6.24; 623/6.28; 623/6.44

(57) ABSTRACT

A multifocal phakic intraocular lens (10) designed to be placed in a phakic eye to correct various refractive disorders such as myopia, hyperopia, astigmatism and presbyopia. The lens (10) which can be positioned within the eye's anterior chamber (62) or posterior chamber (63), consists of a substantially circular optical section (20) having integrally attached either a circular haptic extension (36) or at least two separate haptic extensions (36). When the lens (10) is positioned in the anterior chamber (62), the haptic extensions fixate the lens (10) between the base of the iris (56) and the cornea (72). When the lens (10) is positioned in the posterior chamber (63), the haptic extensions stabilize the lens (10) between the posterior leaf of the iris (56) and the lens capsule (60). In either position, the lens (10) is continuously bathed with the aqueous humour of the eye.

### TECHNICAL FIELD

The invention pertains to the general field of multifocal lenses and more particularly to a multifocal phakic intraocular lens which aids in correcting a variety of refractive disorders such as myopia, hyperopia, astigmatism and presbyopia.

### BACKGROUND ART

Recent advances in human lens technology have advanced the use of phakic intraocular lenses as a method of correcting refractive visual disorders. Generally, a deformable artificial lens is implanted into an eye to remedy myopia, hyperopia or astigmatism. Phakic intraocular lenses can be implanted as a corneal inlay, in the anterior chamber or posterior chamber of the eye. Phakic intraocular lenses have an optical zone portion typically made of silicone or acrylic material, and a supportive element to assist in securing its position within the eye.

In the prior art, phakic intraocular lenses have been limited to a single dioptric power. This version of phakic intraocular lens may be suitable for young individuals who possess the ability to accommodate and adjust their focal distance. However, single-power phakic intraocular lenses in presbyopic individials are unable to provide adequate vision at variable focal distances. Presbyopic individuals with single-power phakic intraocular lenses may have adequate vision for distance, but still require spectacles or contact lenses to see properly for near visual tasks, such as reading. Thus, the single-power phakic intraocular lens does not eliminate the necessity for an external optical appliance to obtain satisfactory vision correction. Eliminating or reducing the necessity of an external optical appliance to see adequately is highly desirable in occupations that involve particulate atmospheric matter, underwater viewing, and sports related activities. In these conditions, the use of an external optical device is impracticable or can limit performance.

Lenses that provide variable focal distances are well known in the art. The most common method of producing multifocal lenses include the placement of curves of varying powers on either the anterior or posterior surface of the lens. These lenses are generally used as a substitute for the natural crystalline lens of the eye. Ion-implantation on selected surface regions of the lens can also provide variable focal powers. However, this method is expensive, difficult to implement and imprecise.

The multifocal phakic intraocular lens of the present invention overcomes these limitations by providing progressive vision correction by utilizing several optical zones located within the lens which is implanted in an eye retaining its natural crystalline lens. Multiple images corresponding to various focal distances are projected on the retina. The brain selects the best-focused image to enable the viewer to see well for far, near and intermediate gazing distances.

A search of the prior art did not disclose any patents that read directly on the claims of the instant invention. However, the following U.S. patents were considered related:

| U.S. PAT. NO. | INVENTOR | ISSUED |
|---|---|---|
| 6,015,435 | Valunin, et al | 18 Jan. 2000 |
| 5,521,656 | Portney | 28 May 1996 |
| 5,225,858 | Portney | 6 Jul. 1993 |
| 5,096,285 | Silberman | 17 Mar. 1992 |

The U.S. Pat. No. 6,015,435 patent discloses a phakic intraocular lens for the correction of visual disorders such as myopia, hyperopia, astigmatism and presbyopia. The lens is made from a biocompatible, elastomeric material such as silicone. The lens includes one or more annular surfaces that protrude from the anterior surface of the lens. The protuberant annular zone contacts the pupillary margin of the iris. As the iris dilates and constricts, contact between the annular zone and iris places a centering force on the implanted lens.

The U.S. Pat. No. 5,521,656 patent discloses an opthalmic lens which has a plurality of alternating power zones with a continuously varying power within each zone, as well as in transition from one zone to another. At least two concentric zones are provided in which the variation from far to near vision correction is continuous. Two versions of the invention are disclosed. In the first version, continuous alternating power variation is accomplished by a continuously changing curvature of the lens' posterior surface, thereby altering the angle of impact of light rays on the eye. In the second version, continuous alternating power variation is accomplished by creating non-homogeneous surface characteristics having refractive material indexes, which continuously vary in the lens' radial direction.

The U.S. Pat. No. 5,225,858 patent discloses a multifocal ophthalmic lens adapted for implantation in the eye or to be disposed on or in the cornea. The lens has an optical axis, a central zone and a plurality of annular zones circumscribing the central zone. Two of the annular zones have a first region with a far vision correction power and a second region with a near vision correction power. The vision correction power between far and near is progressive, and each of the second regions has a segment in which the near vision correction power is substantially constant.

The U.S. Pat. No. 5,096,285 patent discloses multifocal lenses for improving vision. The lens uses at least one diffractive zone located in a defined portion of the surface of a refractive lens to achieve multifocal vision by providing nearly 100% efficiency in the +1 diffractive order. The lens may be used for both contact lenses and intraocular lenses as well as other vision correcting applications.

For background purposes and as indicative of the art to which the invention relates, reference may be made to the following remaining patents found in the search:

| U.S. PAT. NO. | INVENTOR | ISSUED |
|---|---|---|
| 4,199,231 | Evans | 22 Apr. 1980 |
| 4,580,882 | Nuchman | 8 Apr. 1986 |
| 4,585,456 | Blackmore | 29 Apr. 1986 |
| 4,636,049 | Blaker | 13 Jan. 1987 |
| 4,769,033 | Nordan | 6 Sept. 1988 |
| 4,898,461 | Portney | 6 Feb. 1990 |
| 4,917,681 | Nordan | 17 Apr. 1990 |
| 5,019,099 | Nordan | 28 May 1991 |
| 5,089,024 | Christie | 18 Feb. 1992 |
| 5,166,711 | Portney | 24 Nov. 1992 |
| 5,192,317 | Kalb | 9 Mar. 1993 |
| 5,201,763 | Brady, et al | 13 Apr. 1993 |
| 5,270,744 | Portney | 14 Dec. 1993 |
| 5,507,806 | Blake | 16 Apr. 1996 |
| 5,549,668 | O'Donnell | 27 Aug. 1996 |
| 5,702,440 | Portney | 30 Dec. 1997 |
| 5,728,156 | Gupta | 17 Mar. 1998 |
| 5,754,270 | Rehse | 19 May 1998 |
| 5,786,883 | Miller | 28 Jul. 1998 |
| 5,864,378 | Portney | 26 Jan. 1999 |
| 5,919,229 | Portney | 6 Jul. 1999 |
| 6,024,447 | Portney | 15 Feb. 2000 |
| 6,106,553 | Feingold | 22 Aug. 2000 |

Non-patent Documents:

Emanuel Rosen, MD, FRCSE, Christa Gore, MSc, MCOptrom “Staar Collamer posterior chamber phakic intraocular lens to correct myopia and hyperopia”, J. Cataract and Refractive Surgery, Vol. 24, May 1998

### DISCLOSURE OF THE INVENTION

The multifocal phakic intraocular lens is designed to be placed in a phakic eye to correct refractive disorders such as myopia, hyperopia, astigmatism and presbyopia. In its basic design, the lens is comprised of:

A. An anterior surface and a posterior surface; and

B. An optical section which extends from the anterior surface to the posterior surface. The optical section has a substantially circular shape with a perimeter edge having integrally attached a haptic extension.

The optical section is substantially aspheric and has at least two annular zones of refractive power consisting of a central optical zone and a peripheral optical zone. The refractive power of the central optical zone can be substantially focused on the retina for optimal distance vision correction, or focused on the retina for optimal near vision correction. The power of the annular zones are selected to accommodate a range for intermediate, near or distance vision correction. The optical section can also have a toric shape to correct for astigmatism. The lens is designed to be positioned within the posterior or anterior chamber of the eye.

The haptic extension can consist of a single circular member that circumscribes the perimeter edge of the optical section, or can consist of at least two substantially rectangular haptic sections that extend from the perimeter edge of the optical section. When the lens is positioned in the anterior chamber, the haptic extension(s) fixate the lens in the angle between the cornea and the iris. When the lens is positioned in the between the cornea and the iris. When the lens is positioned in the posterior chamber, the haptic extension(s) stabilize the lens between the posterior leaf of the iris and the anterior surface of the natural crystalline lens.

In view of the above disclosure it is the primary object of the invention to produce a multifocal phakic intraocular lens that is dimensioned to be securely positioned within the anterior or posterior chamber of the eye. The lens can be further designed to correct various refractive disorders including myopia, hyperopia, astigmatism and presbyopia.

In addition to the primary object of the invention it is also an object to produce a multifocal phakic intraocular lens that:

provides progressive vision correction between far and near distances, through several optical zones,

allows an eye to retain its natural crystalline lens,

can be manufactured with varying lens designs for correcting various refractive errors,

can be manufactured of a single or combination of compositions to provide varying refractive powers.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a front elevational view of a multifocal phakic intraocular lens (MPIL) having a central optical section bordered by a circular haptic extension.

FIG. 2 is a front elevational view of a MPIL having a central optical section from where extends outward a first haptic extension and a second haptic extension.

FIG. 3 is a front elevational view of a MPIL having an optical section from where extends outward a first undulating haptic extension and a second undulating haptic extension.

FIG. 4 is a cross-sectional view of a MPIL showing the optical section of the lens and a continuous haptic extension.

FIG. 5 is an isometric view of a MPIL wherein the anterior surface of the lens has a toroidal shape to correct astigmatism.

FIG. 6 is a cross-sectional view of a MPIL having a central optical zone that is spherical on the anterior and posterior surfaces, an aspheric peripheral portion and a haptic section extending from the central optical section.

FIG. 7 is a cross-sectional view of a MPIL having a plurality of annular refractive correction zones of varying power on the anterior and posterior surfaces of the central optical section.

FIG. 8 is a cross-sectional view of a MPIL wherein the anterior and posterior surfaces of the lens have a constant aspheric configuration.

FIGS. 9 and 10 are cross-sectional views of a MPIL wherein the optical section of the lens is bordered by a plurality of annular correction zones of various curvatures.

FIG. 11 is a cross-sectional view of a MPIL having a central optical zone with a pin hole aperture.

FIG. 12 is a front elevational view of the MPIL shown in FIG. 11.

FIG. 13 is a cross-sectional view showing a refractive index of the optical zone altered by means of irradiation.

FIG. 14 is a cross-sectional view of an eye wherein the MPIL is inserted and positioned within the posterior chamber of the eye.

FIG. 15 is a cross-sectional view of an eye wherein the MPIL is inserted and positioned within the anterior chamber of the eye.

### BEST MODE FOR CARRYING OUT THE INVENTION

The best mode for carrying out the invention is presented in terms of a preferred embodiment, which incorporates various designs for a multifocal phakic intraocular lens 10 (hereinafter “MPIL 10”). The MPIL is designed and adapted to be inserted into, and positioned within, a mammalian eye 50, which is comprised of a ciliary sulcus 52, a pupillary aperture 54, an iris 56, a lens 58, a lens capsule 60, an anterior chamber 62, a posterior chamber 63, an anterior chamber angle 64, a trabecular meshwork 65, a retina 66, an optic nerve 68, and an optical axis 70, a cornea 72, zonules 74 and a sclera 76.

The MPIL 10, which has a diameter of 9.0 to 12.5 millimeters, is made from a composition selected from the group consisting of biocompatible elastomeric materials which includes silicone, polymethylmethacrylate, acrylate, polyvinylpyrrolidine, polyhydroxymethacrylate, acrylic collagen polymer, blended portions of elastomeric polymers, or similar optically suitable materials. The MPIL 10 can be created with a single, homogenous material having a uniform index of refraction, or with a plurality of non-homogeneous materials having various refractive indices. For optimal utility, the MPIL 10 includes an optical section 20 having annular zones of variable correction power for intermediate, near and distance vision correction.

A full-thickness surgical iridectomy by laser or conventional surgical instruments can be performed before or after placement of the MPIL 10 within the eye. A surgical iridectomy serves to direct aqueous flow from the posterior chamber 63 into the anterior chamber 62 where it exits the eye through the trabecular meshwork 65. Light focused by the MPIL 10 and the natural crystalline lens 58 of the eye 50 is focused on the retina 66 and is transmitted through biochemical processes to the brain by means of the optic nerve 68.

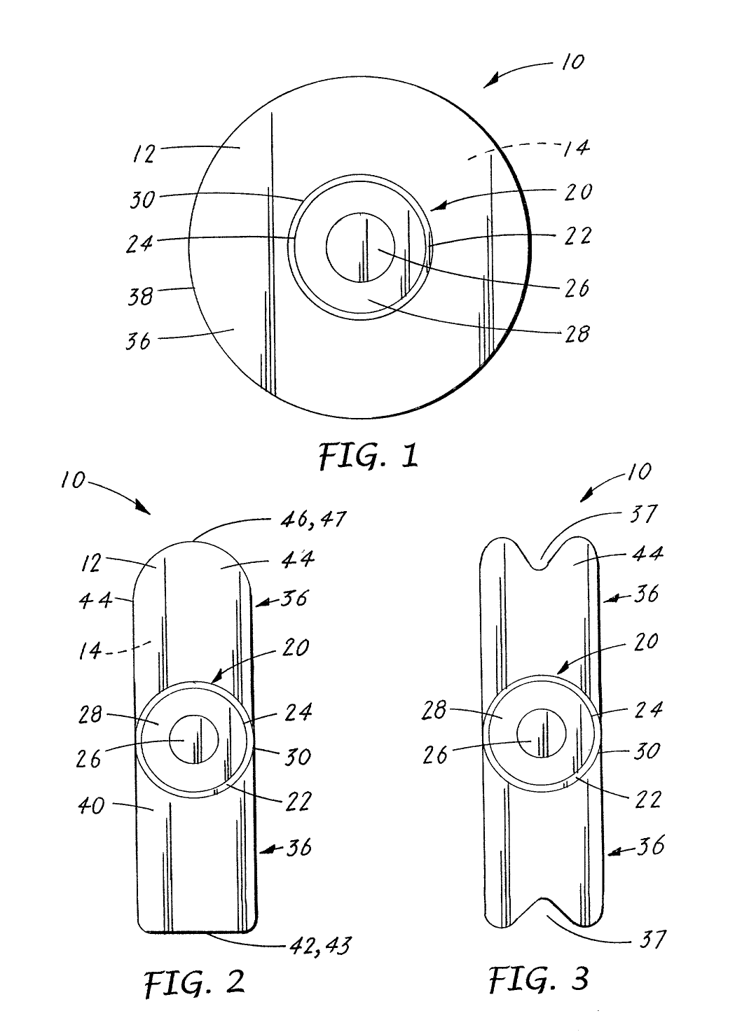

As shown in FIGS. 1-14, the MPIL 10 is comprised of an anterior surface 12, a posterior surface 14 and the optical section 20, which comprises a lens 22 having at least two substantially circular, multifocal optical, annular zones of variable vision corrective power 24.

Within the optical section 20 are located a central optical zone 26 and a peripheral optical zone 28. The optical section 20 extends from the anterior surface 12 to the posterior surface 14, and has a substantially circular shape with a perimeter edge 30 having an integrally attached haptic extension 36. Preferably, a single, continuous haptic extension, which circumscribes the perimeter of the optical section 20 and terminates with an outer edge 38 is utilized, as shown in FIG. 1. As shown in FIG. 2, at least two substantially rectangular haptic extensions 36 can be utilized: a first haptic extension 40 having an outer edge 42, and a second haptic extension 44 having an outer edge 46. The outer edges can have a terminus that is substantially straight 43, as shown on the lower side of FIG. 2, or can be curvilinear 47, as shown on the upper side of FIG. 2. The curvilinear design allows the extensions 36 to be securely positioned between the iris 56 and natural lens 58. As shown in FIG. 3, the rectangular haptic extensions 36 can also be designed with undulating outer edges 37 which allow the extensions to be comfortably fixated in the angle of the anterior chamber 62. The haptic extensions typically have a thickness between 0.03 mm to 0.35 mm.

As shown in FIGS. 1, 2 and 4, the central optical zone 26 has an axial alignment substantially corresponding to the axis of the central retina 66, and is substantially focused on the retina for optimal distance or near vision correction. The peripheral optical zone 28 has annular refractive power zones 24 with progressive or alternating high, low and intermediate vision correction powers. The refractive power zones 24 can be situated on the anterior surface 12, the posterior surface 14, or in a plurality of combinations on the anterior 12 and posterior 14 surfaces of the lens 10.

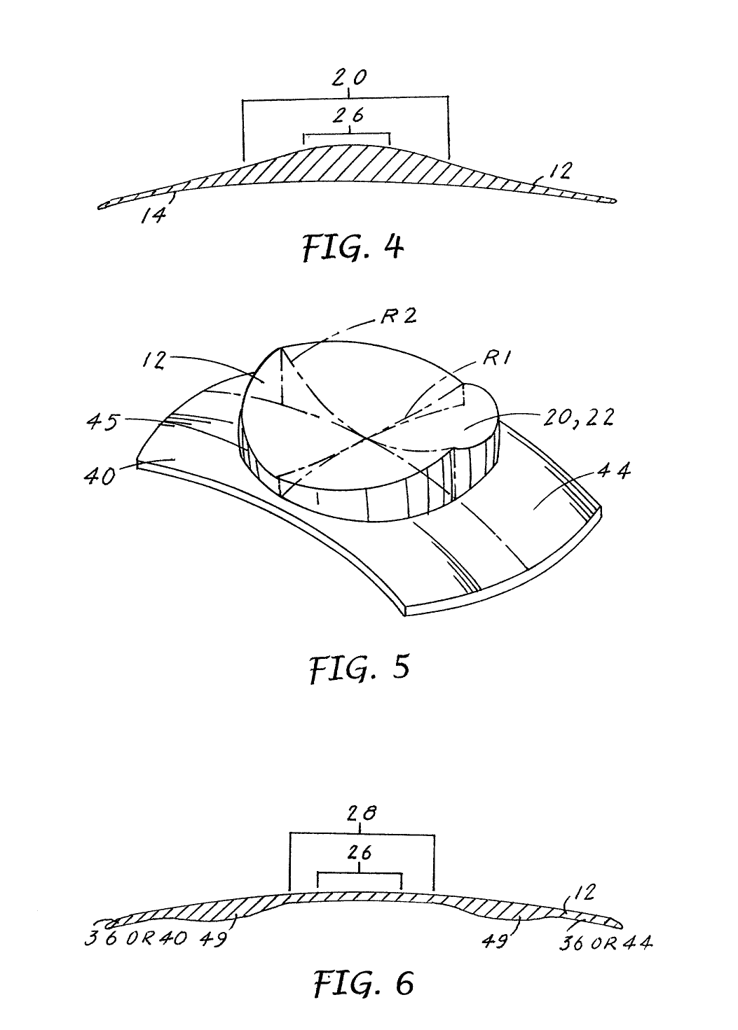

In one design, as shown in FIG. 4, the central optical zone 26 of the optical section 20 has a manufactured central curvature and thickness that determines the refractive powers of the lens for maximizing either distance or near visual acuity. The anterior surface 12 is aspheric and the posterior surface 14 may be either aspheric or spherical. It is desirable for the posterior surface 14 to be formed as a concavity having a radius of curvature less than the radius of curvature of the anterior portion of the natural crystalline lens 58. This configuration minimizes contact between the MPIL 10 and the lens of the eye 50 when the lens is placed in the posterior chamber 63. The concavity of the posterior surface of the MPIL 10 minimizes contact between the iris 56 and the MPIL 10 when it is positioned in the anterior chamber 62 of an eye 50.

As shown in FIG. 5, the optical section can consist of a lens 22 with an anterior surface having a toroidal shape 45 which aids in correcting astigmatism. In this design, the lateral radius R1 is larger than the longitudinal radius R2, and between the two radii is a continuously smooth surface.

As shown in FIG. 6, the central optical zone 26 is spherical on the anterior and posterior surfaces 12,14. The central optical zone 26 can range from 1.5 to 3 mm in diameter. The peripheral portion of the lens 22 is aspheric to provide a progressive refractive change 49. The haptic extension 36 or 40,44 extends from the optical section 20 to position the lens 22 within the anterior or posterior chamber 62,63 of an eye 50.

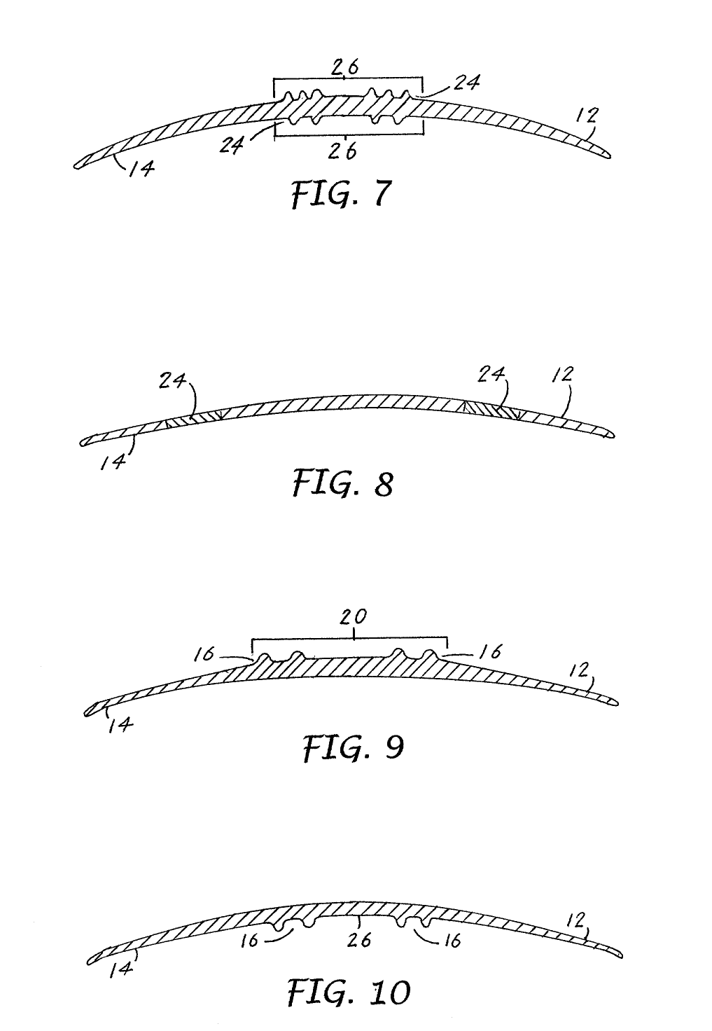

As shown in FIG. 7, a plurality of annular refractive correction zones of varying power are present on the anterior and posterior surfaces 12,14 of the optical section 20 which in combination provide the desired refractive corrections. The posterior surface of the lens has a slightly smaller radius than the anterior surface of a natural crystalline lens 58 to minimize direct contact between the MPIL 10 when it is situated in the posterior chamber 63 of the eye 50.

In FIG. 8, the anterior and posterior surfaces 12,14 of the lens retain a constant aspheric configuration. A plurality of annular refractive zones 24 are accomplished by means of compositions of material with varying refractive indices. Thus, alternating power variation is accomplished by creating refractive zones with non-homogeneous constituent materials with individual refractive indices.

Curves can be placed on the anterior surface 12 near the optical section 20, as shown in FIG. 9, or on the posterior surface 14, as shown in FIG. 10. The curves placed on the anterior surface increase the convexity of the MPIL 10, whereas those on the posterior surface reduce the concavity of the MPIL 10.

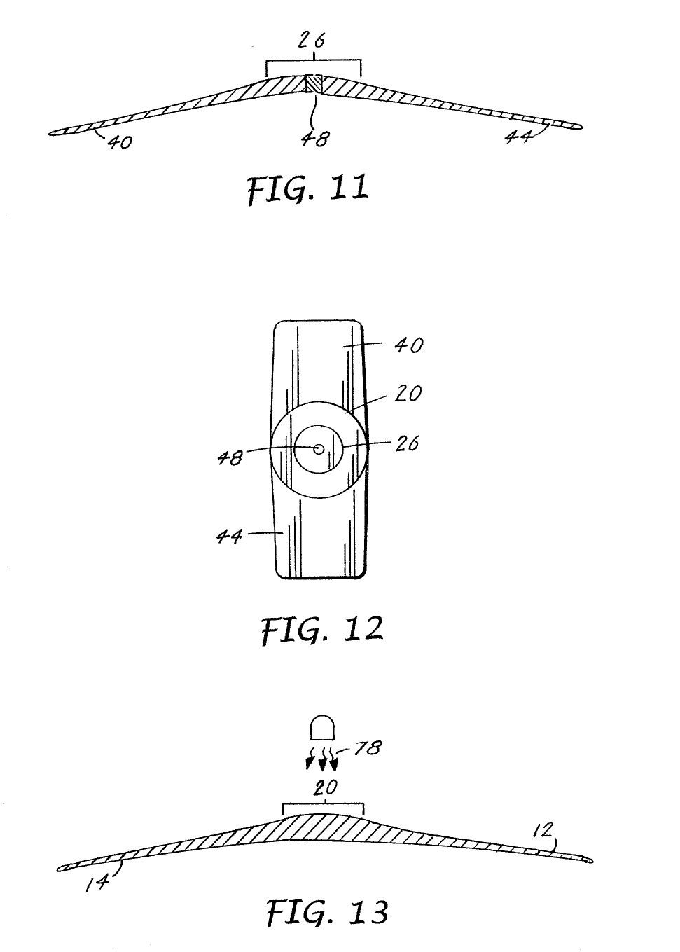

As shown in FIGS. 11 and 12, the central optical zone 26 can further include a pin-size aperture 48 that is between 0.5 and 3.0 millimeters. The advantage of utilizing an aperture 48 is that the “pinhole effect” enhances vision over a wide range of viewing distances.

Additionally, the lens 22 can be comprised of a material having a refractive index capable of changing upon exposure to laser irradiation, or the refractive zones 24 may vary with laser irradiation. As shown in FIG. 13, the refractive index of the optical zone 20 is altered by means of irradiation 78. The MPIL 10 in this design, is manufactured with a material which changes its refractive index when exposed to the laser irradiation. Medical grade optical materials which are susceptible to refractive changes when exposed to laser irradiation are known in the art and therefore are not described herein. Laser irradiation originating from a source external to the eye can be transmitted sequentially through the cornea and aqueous humor, and is ultimately absorbed by the MPIL 10 without injury to other intraocular structures. The number and pattern of laser treatments to the MPIL 10 can be selected to achieve variable zones of refractive power.

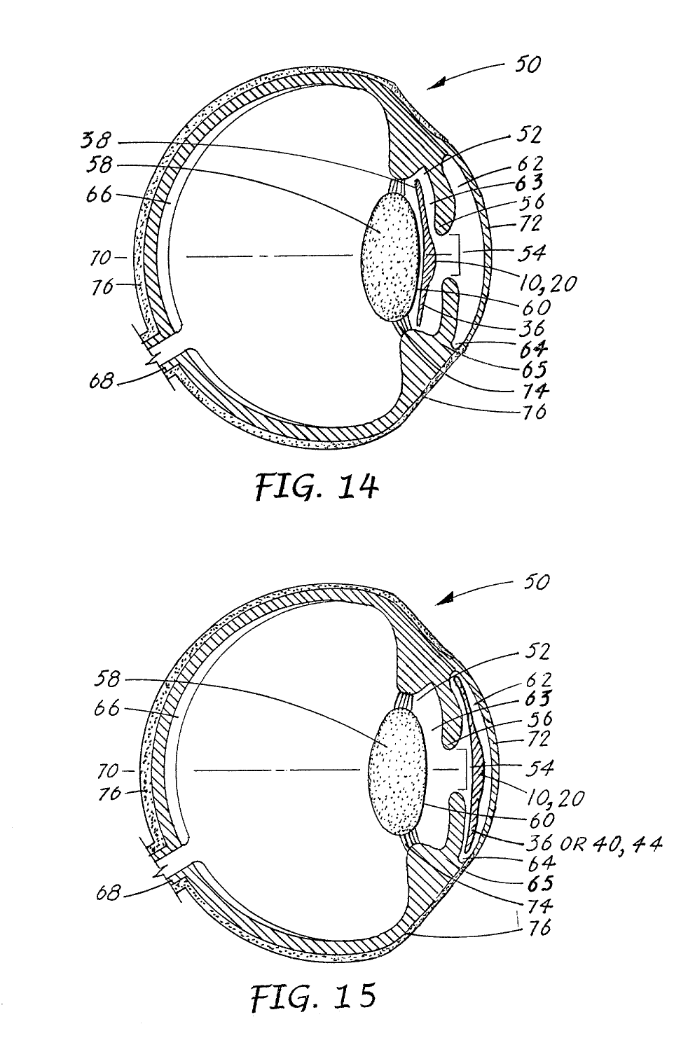

As shown in FIG. 14, the MPIL 10 is inserted into and positioned within the posterior chamber 63 of the eye 50, with the optical section 20 positioned behind the pupillary aperture 54. Either the single haptic extension 36 or the multiple extensions 40,44 float freely between the posterior leaf of the iris 56 and the lens capsule 60, with the outer edges 38 of the haptic extension 36 located adjacent to the respective ciliary sulcus 52.

As shown in FIG. 15, the optical section 20 is positioned in front of the pupillary aperture 54. The haptic extension 36 or 40,44 fixate in the anterior chamber 62 between the base of the iris 56 and the cornea 72. A full-thickness surgical iridectomy by laser or conventional surgical instruments can be performed before or after placement of MPIL 10 within the eye to prevent pupillary block glaucoma. Light focused by the MPIL 10 and the natural crystalline lens 58 is focused on the retina 66 and is transmitted through biochemical processes to the brain by means of the optic nerve 68.

While the invention has been described in complete detail and pictorially shown in the accompanying drawings it is not to be limited to such details since many changes and modifications may be made to the invention without departing from the spirit and the scope thereof Hence, it is described to cover any and all modifications and forms which may come within the language and scope of the claims.

1. A multifocal phakic intraocular lens adapted to be positioned within a mammalian eye comprising a ciliary sulcus, a pupillary aperture, an iris, a lens, a lens capsule, an anterior chamber, a posterior chamber, an anterior chamber angle, a trabecular meshwork, a retina, an optic nerve, an optical axis, a cornea, a zonules and a sclera, said lens comprising:
a) an anterior surface,
b) a posterior surface, and
c) an optical section extending from the anterior surface to the posterior surface and having a substantially circular shape with a perimeter edge integrally attached to a haptic extension.

2. The lens as specified in claim 1 wherein the anterior and posterior surfaces are substantially aspheric.

3. The lens as specified in claim 1 wherein the optical section has at least two annular zones of refractive power.

4. The lens as specified in claim 1 wherein said haptic extension circumscribes the perimeter of the optical section and terminates with an outer edge.

5. The lens as specified in claim 1 wherein said haptic extension is comprised of at least two substantially rectangular haptic extensions each terminating with an outer edge.

6. The lens as specified in claim 5 wherein said rectangular haptic extensions have an undulating outer edge.

7. The lens as specified in claim 1 wherein said lens is made of a composition selected from a group consisting of biocompatible elastomeric materials which includes silicone, polymethylmethacrylate, acrylate, polyvinylpyrrolidine, polyhydroxymethacrylate, acrylic collagen polymer, blended portions of elastomeric polymers, and other elastomeric compositions.

8. The lens as specified in claim 3 wherein the annular zones of refractive power comprises a central optical zone and a peripheral optical zone and consist of materials of varying refractive index.

9. The lens as specified in claim 8 wherein the power of the central optical zone is substantially focused on the retina for optimal distance vision correction.

10. The lens as specified in claim 8 wherein the power of the central optical zone is substantially focused on the retina for optimal near vision correction.

11. The lens as specified in claim 3 wherein the power of at least two annular zones have a power range for intermediate, near and distance vision correction.

12. The lens as specified in claim 1 wherein said lens is positioned within the posterior chamber of an eye.

13. The lens as specified in claim 1 wherein said lens is positioned within the anterior chamber of an eye.

14. The lens as specified in claim 1 wherein said haptic extensions have a thickness between 0.03 to 0.35 mm.

15. The lens as specified in claim 1 wherein said haptic extension consist of an annular extension which circumscribes the optical zone.

16. The lens as specified in claim 1 wherein the diameter of said lens is between 9.0 to 12.5 mm.

17. The lens as specified in claim 8 wherein the central optical zone and the peripheral optical zone have a substantially circular shape.

18. The lens as specified in claim 17 wherein the central optical zone is spherical and the peripheral optical zone is aspheric.

19. The lens as specified in claim 8 wherein the optical section is toric shaped to correct for astigmatism.

20. The lens as specified in claim 1 wherein the posterior surface is formed as a concavity having a radius of curvature less than the radius of curvature of the anterior portion of the natural crystalline lens.

21. A multifocal phakic intraocular lens adapted to be positioned within a mammalian eye comprising a ciliary sulcus, a pupillary aperture, an iris, a lens, a lens capsule, an anterior chamber, a posterior chamber, an anterior chamber angle, a trabecular meshwork, a retina, an optic nerve, an optical axis, a cornea, a zonules and a sclera, said lens comprising:
a) an anterior surface,
b) a posterior surface, and
c) an optical section extending from the anterior surface to the posterior surface and having at least two annular zones of refractive power, and a substantially circular shape with a perimeter edge integrally attached to a haptic extension.

22. The lens as specified in claim 21 wherein said annular refractive power zones are situated on the anterior surface of the lens.

23. The lens as specified in claim 21 wherein said annular refractive power zones are situated on the posterior surface of the lens.

24. The lens as specified in claim 21 wherein the optical section is toric shaped to correct for astigmatism.

25. The lens as specified in claim 21 wherein said annular refractive power zones are situated in a plurality of combinations on the anterior and posterior surface of the lens.

27. The lens as specified in claim 21 wherein the plurality of annular refractive power zones have varying index of refraction which provide a plurality of refractive zones.

28. The lens as specified in claim 21 wherein the lens is comprised of a material having a refractive index capable of changing upon exposure to laser irradiation.

29. The lens as specified in claim 21 wherein the plurality of annular power refractive zones vary with laser irradiation.

30. The lens as specified in claim 21 wherein the central optical zone further comprises a pinhole aperture.
